(19) United States
(12) Patent Application Publication
(10) Pub. No.: US 2006/0269556 A1
(43) Pub. Date: Nov. 30, 2006

(54) Mast cell activation using siglec 6 antibodies

(76) Inventors: Karl Nocka, Harvard, MA (US); Sudhir Rao, Somerville, MA (US)

Correspondence Address: MORGAN LEWIS & BOCKIUS LLP, 1111 PENNSYLVANIA AVENUE NW, WASHINGTON, DC, 20004, US

(21) Appl. No.: 11/405,695

(22) Filed: Apr. 18, 2006

Related U.S. Application Data
(60) Provisional application No. 60/672,012, filed on Apr. 18, 2005.

(51) Int. Cl. A61K 39/395; G01N 33/574; C07K 14/52; C07K 14/53; C07K 16/30; C07K 16/46

(52) U.S. Cl. 424/155.1; 530/387.3; 530/351; 424/178.1; 435/7.23

(57) ABSTRACT

The present invention provides a method of modulating mast cell function using Siglec 6 antibodies or fragments thereof. The present invention also provides methods of treating or preventing diseases or disorders associated with mast cell function.

### RELATED APPLICATIONS

This application claims the benefit of U.S. Provisional Application 60/672,012, filed Apr. 18, 2005, which is incorporated by reference in its entirety.

### FIELD OF THE INVENTION

The present invention is related to methods of modulating mast cell functions. Specifically, the present invention provides methods of identifying agents and methods of treating and/or preventing disorders and diseases associated with mast cell functions.

### BACKGROUND OF THE INVENTION

The Role of Mast Cells

Mast cells play an important role in triggering the body's immune response. They are involved in various diseases and disorders such as allergic diseases, inflammatory diseases, autoimmune disorders, and hypersensitivity disorders. Mast cells are a normal component of connective and mucosal tissues of the various organs of the body. They are located in these tissues because these tissues are the main entry points for infective organisms, allergens and other noxious chemicals that trigger the body's immune response.

Mast cells are produced by hematopoietic stem cells. In response to signals, mast cell progenitors travel through the blood vessels, exiting and moving into tissue until they reach their target site. In the target tissue, they mature into the form recognized as mast cells. Receptors on the surface of mast cells are the means by which these cells receive signals from the surrounding tissue and from substances that leave the blood stream to enter tissues. The best-characterized mast cell receptors are those for IgE (FcεRI), the “allergy” immunoglobulin produced by immune system cells, but there are numerous other kinds of receptors. Inappropriate activation of mast cells through any of these receptors may be involved in mast cell activation disorder. A defect in mast cell receptors that inhibit activation may also be involved in inappropriate mast cell activation.

Mast cells contain, or can produce a wide array of chemicals and immune system communication molecules, allowing each mast cell to react to changes in the tissue around it. Examples of such chemicals, also known as mediators, include but are not limited to histamines, proteases, prostaglandin D2 (PGD2), leukotriene C4, interleukin-5 (IL-5), and interleukin-1β (IL-1β). When antigen binds to IgE on the surface of mast cells, a variety of mediators are released resulting in increased vascular permeation, vasodilation, bronchial and visceral smooth muscle contraction, and local inflammation. The binding of antigen to IgE on mast cells can trigger an immediate hypersensitivity reaction. In the most extreme form of immediate hypersensitivity reaction known as anaphylaxis, mediators released from mast cells can restrict airways to the point of asphyxiation. Atopic individuals, who are prone to develop strong immediate hypersensitivity responses, may suffer from asthma, hay fever or chronic eczema. These individuals possess higher than average plasma IgE levels.

In the skin, the release of these mediators produces urticaria, or hives, and when the stimulus for activation of mast cells is unknown and the patient has repeated episodes of urticaria they may be diagnosed with chronic idiopathic urticaria. Swelling in deeper tissues produces a condition known as angioedema.

Siglec 6

Sialic acid-binding immunoglobulin superfamily lectins (Siglecs) are differentially expressed on hematopoietic cells. They consist of a family of structurally and functionally related cell surface receptors that bind to sialic acid containing carbohydrate groups of glycoproteins and glycolipids as ligands. Several members have been identified in the human genome including sialoadhesin (Siglec 1), CD22 (Siglec 2), CD33 (Siglec 3), myelin-associated glycoprotein (MAG; Siglec-4), and Siglecs 5-11 which are closely related to CD33 (Angata et al., 2002, J. Biol. Chem. 277, 24466-24474; Angata et al., 2002, Biochim. Biophys. Acta, 1572, 294-316). In the murine genome, eight Siglecs have been identified including orthologs for sialoadhesin, CD22, CD33, MAG, and Siglec 10 (mSiglec G), and three other CD33 related siglecs (Siglecs E, F, and H) that have no clear cut human ortholog (Crocker et al., 2002, Curr. Opin. Struct. Biol. 12, 609-615).

Structurally, these receptors have between two to seventeen extracellular Ig-like domains, a transmembrane domain, and a cytoplasmic tail. The distal most N-terminal Ig domain is composed of a characteristic “V-set” domain that is responsible for binding sialic acid containing ligands. Some Siglecs may have one or more immune receptor tyrosine-based inhibitory motifs (ITIMs) in their cytoplasmic tail which are believed to be involved in the regulation of receptor mediated signal transduction (Crocker et al., 2001, Trends Immunol. 22, 609-615).

Patel et al. reported the expression and cloning of Siglec 6/OB-BP1 (1999, J. Biol. Chem. 274, 22729-22738). Siglec 6 is a novel leptin-binding protein that has a high level of amino acid identity with other members of the Siglec family (59% with Siglec 5 and 63% with Siglec 3). Patel et al. demonstrated that Siglec 6 selectively bound Neu5Acα2-6BaINAca(sialyl-Tn). Using the extracellular domain of Siglec 6 as an immunogen, Patel et al. generated a specific mouse monoclonal antibody against Siglec 6 (10F10). The antibody was used to confirm the strong expression of Siglec 6 in the trophoblasts of placenta. Siglec 6 was also shown to be exclusively expressed on B cells.

### SUMMARY OF THE INVENTION

The invention provides a method of modulating mast cell functions using Siglec 6 antibodies or fragments thereof. In particular, the invention provides a method of inhibiting mast cell functions by contacting the mast cells with Siglec 6 antibodies or fragment thereof.

In one embodiment, the invention provides a method for treating a disease or disorder characterized by mast cell dysfunction. An effective amount of Siglec 6 antibody which inhibits mast cells function is administered to a subject diagnosed with the disease or disorder. The disease or disorder may, for example, be asthma, urticaria, or atopic dermatitis.

In another embodiment, the invention also provides a method for selecting agents useful for modulating mast cell activation. The method comprises incubating mast cells expressing Siglec 6 with a test agent and determining whether the test agent modulates at least one Siglec 6 dependent mast cell function. Examples of such agents include proteins, peptides, small molecules, vitamin D derivatives, as well as carbohydrates. Agents that modulating mast cell activation may be useful for inhibiting mast cell function.

In one embodiment, the agent is an antibody or a fragment thereof, for example, the Siglec 6 antibody or a fragment thereof. In another embodiment, the agent may be a small molecule or a pepetide mimetic that inhibits mast cell function.

### BRIEF DESCRIPTION OF THE DRAWINGS

FIG. 1 depicts the structural differences of members of the Siglec family.

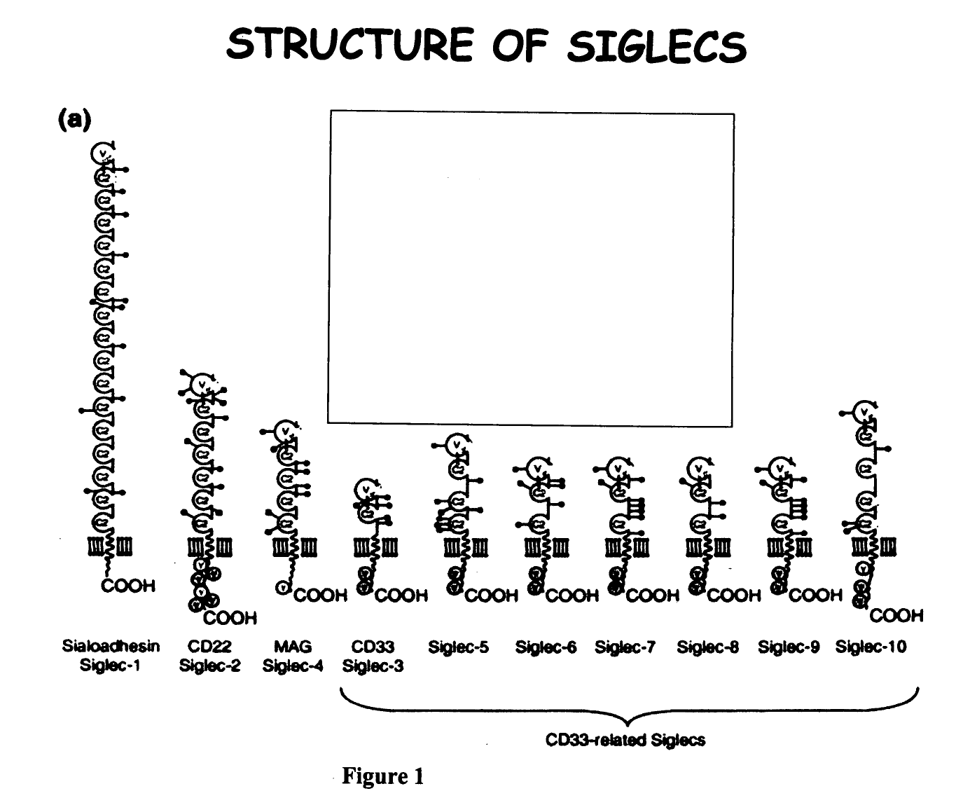

FIG. 2 is an electronic Northern (E-Northern) analysis of Siglec 6 mRNA expression in a number of normal human tissues and inflammatory cell populations.

FIG. 3 shows the inhibition of human mast cell activation induced by the IgE-antigen complex by monoclonal anti-Siglec 6 antibodies in a dose dependent manner.

FIG. 4 shows inhibition of human mast cell activation induced by complement-derived anaphylatoxins C3a by monoclonal anti-Siglec 6 antibodies in a dose dependent manner.

FIG. 5 illustrates the timecourse of Siglec 6 expression knockdown by single stranded interfering RNA (RNAi) specific to Siglec 6.

FIG. 6 shows the inhibitory effect of Siglec 6 crosslinking on mast cells β-hexosaminidase release after the cells were treated either with single stranded Siglec 6 RNAi or control.

### DETAILED DESCRIPTION OF THE INVENTION

1. General Description

The present invention is based in part on the finding that Siglec 6 has a mast cell specific expression profile. The inventors discovered this from a biochip experiment with cultured human mast cells (HuMC). A comparison with other human hematopoietic cells, as well as human tissues in the database revealed that Siglec 6 possesses a very restricted expression pattern. This was also confirmed with cord blood derived mast cells (Nakajima et al. 2001 Blood, 98(4), 1127-1134).

The inventors tested a monoclonal antibody to human Siglec 6 for its ability to modulate the activation of mast cells triggered by IgE and antigen. It was found that the addition of an antibody to Siglec 6 reduced degranulation of HuMC triggered by IgE and antigen. An isotype matched control antibody had no effect on activation. The effect of the Siglec 6 was dose dependent and also the magnitude of the inhibition was dependent on the dose of the stimulus (antigen). High doses of antigen (10 ng/ml) were only partially inhibited by the Siglec 6 antibody, while moderate doses could be completely inhibited.

2. Definitions

As used herein, the term “mast cells” includes all tissue mast cells.

As used herein, the term “cytokines” are proteins that regulate and coordinate many of the activities of the immune system. Cytokines produced by mononuclear phagocytes have been called monokines, while those produced by lymphocytes have been called lymphokines. Most of the cytokines have numbered names while others have trivial names, such as interferon (IFN) and tumor necrosis factor (TNF) (Abbas, et al., 2000 Cellular and Molecular Immunology, 4^{th} ed., W. B. Saunders Co., New York, N.Y., pp. 235, 486).

As used herein, the term “antibody” refers to polyclonal antibodies, monoclonal antibodies, humanized antibodies, single-chain antibodies, and fragments thereof such as Fab, F(ab′)₂, Fᵥ, and other fragments which retain the antigen binding function of the parent antibody.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fᵥ, and others which retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term “humanized antibodies” means that at least a portion of the framework regions of an immunoglobulin are derived from human immunoglobulin sequences.

As used herein, the term “stimulated mast cell” means a mast cell in an activated state which is characterized by, or proximally leads to, degranulation and/or release of mediator from the cell.

As used herein, the term “immunologically stimulated mast cell” means a mast cell which becomes stimulated by binding of antigen to IgE on the cell surface. Mast cell immunologic stimulation also includes experimental immunological stimulation achieved by contacting mast cells with antibodies to IgE, which results in the cross-linking of attached FcεRI receptors on the mast cell. Immunological stimulation may also encompass IgG mediated crosslinking of Fcγ receptors as well.

3. Specific Embodiments

Mast Cells and Their Functions

The present invention provides a method of modulating mast cell functions using anti-Siglec 6 antibodies or fragments thereof. The mast cell functions to be modulated include, but are not limited to, activation, growth, maturation, proliferation, migration, survival, apoptosis, degranulation, and mediator release. One embodiment of the invention involves using Siglec 6 antibodies or fragments thereof to inhibit stimulated human mast cells (HuMC) from releasing mediators. Another embodiment involves the use of Siglec 6 antibodies to modulate HuMC functions of survival and proliferation by interfering with the apoptotic processes of the cells.

Mast cells are generally considered to be long lived cells. Survival, growth and differentiation of mast cells are regulated by cytokines such as kit ligand (KL, SCF, steel factor). Mast cells have the capacity to survive the activation induced degranulation process, and to subsequently regranulate, enabling them to be activated again. Multiple rounds of mast cell activation may underlie the mechanism regulating the reoccurring inflammatory attacks of allergic patients during a pollen season. The possibility of regulating the longevity of activated mast cells could thus provide a treatment for mast cell mediated inflammatory disorders, such as allergies and asthma.

It has been shown that KL critically regulates the migration and survival of mast cell precursors, promotes the proliferation of both immature and mature mast cells, enhances mast cell maturation, directly induces secretion of mast cell mediators, and can regulate the extent of mediator release in mast cells activated by IgE-dependent mechanisms. (Galli et al., 1993, Am. J. Pathology 142:965-974). In addition, KL is one of the hematopoietic growth factors involved in the production of blood cells from bone marrow precursors. (Kaushansky, 1992, Proteins: Structure, Function, and Genetics 12:1-9).

Work has suggested that rhKL and anti-IgE may act on human mast cells through a common pathway to increase free cystolic calcium, and that this effect can be similarly modulated by various drugs. (Columbo et al., 1994, Biochem. Pharmacol 47:2137-2145). Other work has shown that KL potentiates the release of cytokines in response to IgE cross-linking, and perhaps does not act to stimulate release directly. (Bischoff and Dahinden, 1992, J. Exp. Med. 175:237-244). In contrast, work with rrKL (recombinant rat KL) in mice indicates that chronic treatment with rrKL induces mast cell hyperplasia, but does not increase the severity of IgE-dependent anaphylactic reactions. (Ando et al, 1993, J. Clin. Invest. 92:1639-1649).

Additionally, mast cells comprise a normal component of the connective tissue that plays an important role in hypersensitivity and inflammatory reactions by secreting a large variety of chemical mediators from storage sites in their granules upon stimulation. Mast cells, and their circulating counterparts, the basophils, possess surface receptors known as FcεRI. These receptors are specific for antibody ε heavy chains. Several mast cell surface receptors different from known FcεRI subunits have been identified on the rat mucosal type mast cell line RBL-2H3, mainly by specific monoclonal antibodies (mAb), and shown to modulate the FcεRI-mediated secretory response. For example, G63, a mAb that binds a surface glycoprotein named MAFA, or mast cell function-associated antigen, was shown to inhibit both the FcεRI-induced signaling cascade upstream to PLCγ1 activation (e.g. phosphatidylinositide hydrolysis products and transient rise in the cytoplasmic concentration of free Ca²⁺ ions), and the culminating secretion of the cells' granule contents (Ortega and Pecht, 1988, J. Immunol., 141: 4324-4332). MAb G63 inhibitory effect required MAFA clustering, and was not due to interference with IgE-FcεRI interactions. Still, cross-linking of FcεRI-IgE complexes by multivalent antigen also led to co-clustering of the MAFA with the aggregated FcεRI and in the enhancement of its internalization (Ortega et al., 1991, Int. Immunol., 3: 333-342).

Mast cells and basophils are immunologically activated by aggregation of IgE molecules bound to the FcεRI with multivalent antigen. Cellular response can also be induced by directly cross-linking the FcεRI, for example, with anti-receptor antibodies. Clustering of the FcεRI on mast cells and basophils by either IgE and polyvalent antigen or directly by specific monoclonal antibodies, initiates a cascade of biochemical processes coupling to the cells secretory response. These include: (i) the activation of receptor-associated protein tyrosine kinases (Eiseman and Bolen, 1992, Nature, 355: 78-80) and phosphatases (Hampe and Pecht, 1994, FEBS Lett., 346:194-198) causing a transient increase in tyrosine phosphorylation of several cellular proteins (Benhamou et al., 1990, Proc. Natl. Acad. Sci. USA 87:5327-5330 and 1992, J. Biol. Chem., 267:7310-7314); (ii) an increase in phosphoinositides hydrolysis (Beaven et al., 1984, J. Biol. Chem., 259: 7137-7142) resulting from PLCγ1 activation (Li et al., 1992, Mol. Cell. Biol. 12: 3176-3182); and (iii) the rise in the intracellular concentration of free calcium ions (Beaven et al., 1984, J. Biol. Chem., 259: 7129-7136). The final response to this stimulus is the secretion of granule-stored mediators and the de novo synthesis and secretion of mediators of inflammation and the allergic response, including histamine, serotonin, arachidonic acid metabolites, i.e. leukotrienes (Ortega et al., 1989, Eur. J. Immunol., 19: 2251-2256), and prostaglandins, as well as several cytokines (Bradding et al., 1993, J. Immunol., 151:3853-3865; Galli et al., 1991, Curr. Opin. Immunol., 3: 865-873).

Clustering of the FcεRI present in their plasma membranes initiates a signaling cascade culminating in the secretion of mediators of immediate-type allergic reactions (Schwartz, 1994, Cur. Opin. Immunol. 6: 91-97). The molecular mechanism of signal transduction initiated by FcεRI clustering has been studied intensely (Ravetch et al, 1991, Annu. Rev. Immunol. 9:457-492); Holowka et al, 1992, Cell Signal. 4:339-349; Benhamou et al, 1992, Immunol. Today 13:195-197; Beaven et al, 1993, Immunol Today 14:222-226). The β and γ subunits of the clustered FcεRI were found to interact with src family protein tyrosine kinases (PTK) and cause biochemical event in their cascade. The differential control of these PTK by the β and γ chains of the receptor has also been described recently (Jouvin et al, 1994, J. Biol. Chem. 269:5918-5925). The following steps downstream involve recruitment of PTK of the syk family, activation of phospholipase Cγ, which in turn leads to hydrolysis of phosphatidyl inositides and production of inositol triphosphate and diacylglycerol. The former product causes the transient increase in free cytosolic calcium ion concentration while the latter is involved in activation of protein kinase C (Ravetch et al, 1991, Annu. Rev. Immunol. 9:457-492; Holowka et al, 1992, Cell Signal. 4:339-349; Benhamou et al, 1992, Immunol. Today 13:195-197; Beaven et al, 1993, Immunol. Today 14:222-226).

At least two types of mast cells have been defined in rodents which differentiate from a common precursor to produce the serosal (or connective tissue type) and the mucosal type mast cells. Both phenotypes express FcεRI on their cell membrane; however, they respond differently to secretagogues and inhibitors. For example, only serosal-type mast cells are triggered by cationic peptides (including the complement-derived peptides C3a and C5a; venom-peptides, e.g., mastoparan, mellitin; neuropeptides) or polyamines; mucosal mast cells are non-responsive to these stimuli (Mousli et al, 1994, Immuno. Pharmacology 27:1-11).

Mast cells may also be activated by mechanisms other than cross-linking FcεRI, such as in response to mononuclear phagocyte-derived chemokines, to T cell-derived cytokines and to complement-derived anaphylatoxins such as peptides C3a and C5a. C3a is a 9 kilodalton protein fragment released from complement C3 by the action of C3a convertases. C5a is 11,000-dalton fragment released by the action of C5 convertases. Both peptides are capable of binding to G-protein coupled receptors on mast cells and basophils, resulting in the release of histamines and other mediators of anaphylaxis. For this reason, C3a and C5a are termed anaphylotoxins. In rodents, C3a and C5a can cause activation of serosal type mast cells that results in the release of histamine or serotonin and several other inflammatory mediators including proteases, lipid mediators and several cytokines Mast cells may also be recruited and activated by other inflammatory cells or by neurotransmitters which serve as links to the nervous system.

Mediators Released from Mast Cells

When antigen binds to IgE molecules attached to the surface of mast cells, a variety of mediators are released which give rise to increased vascular permeation, vasodilation, bronchial and visceral smooth muscle contraction, and local inflammation. In the most extreme form of immediate hypersensitivity reaction known as anaphylaxis, mediators released from mast cells can restrict airways to the point of asphyxiation.

Mediators released from mast cells may be divided into two broad classes, pre-formed or secretory granule associated mediators and nonpreformed or newly synthesized mediators. The pre-formed mediators include biogenic amines, most notably histamine. The pre-formed mediators also comprise granule macromolecules such as proteoglycans, most notably heparin and chondroitin sulfate E; chemotactic factors such as eosinophil and neutrophil chemotactic factors of anaphylaxis; and enzymes such as proteases, tryptase, chymase, cathepsin G-like enzyme, elastase, carboxypeptidase A and acid hydrolases. The nonpreformed mediators or newly synthesized mediators include products of arachidonic acids such as but not limited to leukotrienes, prostaglandins, thromboxanes and platelet activating factor (PAF). Some examples of leukotrienes include LTC4, LTD4, LTE4, and LTB4. Examples of prostaglandins and thromboxanes include prostaglandin D₂ (PGD2), prostaglandin F2 (PGF2) and thromboxane A2 (TXA2).

Cytokines including chemokines are another class of mediators. They are produced by mast cells upon IgE-mediated activation, or by other cells, including recruited Th2 lymphocytes. These cytokines are predominantly responsible for the late phase reaction which begins two to four hours after elicitation of many immediate hypersensitivity reactions. Some examples of cytokines include but are not limited to interleukin-1b (IL-1b), IL-3, IL-5, GMCSF, and tumor necrosis factor alpha (TNF-α). Examples of chemokines include MCP-1, mip1b, IL-8, and RANTES.

TNF-α may exist in the mast cells as preformed stores, or may represent a newly synthesized product released over a period of hours. TNF-α contributes to the inflammatory process by releasing histamine and by inducing endothelial expression of E-selectin, an adhesion molecule which is critically required for the rapid adhesion of neutrophils, T cells, monocytes, and other leukocytes to endothelial cells.

Mediators released from human mast cells are central to the pathophysiology of allergy, asthma and anaphylaxis. In particular, mast cells and their release of histamine and other mediators play an important role in the symptomatology of asthma and other human diseases. During the early phase of human lung hypersensitivity reactions upon exposure to antigen (i.e., pollens, cats, etc.), mast cells release of mediators are the major source of histamine, and newly synthesized lipid products of arachidonic acid metabolism: prostaglandin D₂ and leukotriene C₄. These mediators produce immediate breathlessness, which subsides in one hour but returns within 2-4 hours (the “late phase” response). Attesting to their primal role in hypersensitivity responses, human lung mast cells are characterized by mRNA generation, protein synthesis and release of so-called Th2 cytokines within these first few hours of activation. These cytokines including IL-5, and IL-13 are believed to be central to the evolution of chronic allergic/asthmatic states. In the lung, only mast cells are a source of histamine. Thus, histamine release is a distinct marker of mast cell activation and behavior. For a review of the role of mast cells in inflammatory responses in the lung, see Schulman, Critical Reviews in Immunology, 13(1):35-70 (1993), the entire disclosure of which is incorporated herein by reference.

Biological and Pharmaceutical Agents Modulating Mast Cell Functions

Since mast cells play an important role in the pathophysiology of allergy, asthma and anaphylaxis and other diseases and disorders, there has been a considerable interest in developing biological and pharmaceutical agents useful in modulating the function of mast cells, in particular, the inhibition of mediator release by mast cells. Mast cell inhibitors that either alter the function of mast cells or inhibit the release of inflammatory mediators from activated mast cells have been identified. Examples of mast cell degranulation inhibitors include but are not limited to picetannol, benzamidines, tenidap, tiacrilast, disodium cromoglycate, Iodoxamide ethyl, and Iodoxamide tromethamine.

Examples of mast cell mediator inhibitors include agents that block the release or secretion of histamine, such as FK-506 and quercetin; antihistamines such as diphenhydramine; and theophylline. Other mast cell inhibitors include serine protease inhibitors, such as α-1-protease inhibitor; metalloprotease inhibitors; lisofylline; TNFR-FE; benzamidine; amiloride; and bis-amidines such as pentamidine and bis(5-amidino-2-benzimidazolyl)methane.

The present invention provides a novel agent for modulating the functions of mast cells. The present invention provides Siglec 6 antibodies and fragments thereof as pharmaceutical agents for modulating the functions of mast cells. In particular, the inventors of the present invention have demonstrated that Siglec 6 antibodies, by cross-linking the Siglec 6 receptors on the surface of human mast cells, exert an inhibitory effect of HuMC. The inventors have also shown that Siglec 6 cross-linking inhibits HuMC activation induced by C3a. Siglec 6 antibodies can reduce degranulation of HuMC induced by IgE and antigen. Thus, the present invention discloses a method of treating and preventing diseases associated with mast cell function. The mast cell function could be due to inappropriate production of IgE, imbalance of antibody types, Th2 T cell responses, or other responses.

Siglec 6 Antibodies

The present invention provides pharmaceutical compositions comprising Siglec 6 antibodies and fragments thereof for modulating, specifically inhibiting, mast cell functions and treating and preventing diseases associated with mast cell function. The mast cell function may be a response to the bodies inappropriate production of IgE, imbalance of antibody types or Th2 T cell responses, or other aberrant processes. The Siglec 6 antibodies used in the present invention will selectively bind to Siglec 6 and will not bind or only will bind weakly to non-Siglec 6 proteins. In one embodiment, the Siglec 6 antibodies will specifically bind to Siglec 6. The term “specifically binds” means that the antibody predominantly binds to Siglec 6.

Anti-Siglec 6 antibodies that are useful in the present invention include but are not limited to functionally active fragments, derivatives, or analogs. They may be monoclonal, polyclonal, bispecific, multivalent, chimeric, human, humanized, single chain, anti-idiotypic (anti-Id) antibodies, recombinant antibodies, Fab fragments and F(ab′) fragments, fragments produced by a Fab expression library and epitope-binding fragments of any of the above. The fragments may contain the antigen binding domain and/or one or more complementarity determining regions (CDRs) of these antibodies. These antibodies can be from any source, e.g., rat, dog, cat, pig, horse, mouse or human.

Various methods for the preparation of antibodies are well known in the art. For example, antibodies may be prepared by immunizing a suitable mammalian host using a Siglec 6 protein, peptide, or fragment, in isolated or immunoconjugated form (Harlow, 1989, Antibodies, Cold Spring Harbor Press, NY). In addition, fusion proteins of Siglec 6 may also be used, such as a Siglec 6 GST-fusion protein. Cells expressing or overexpressing Siglec 6 may also be used for immunizations. Similarly, any cell engineered to express Siglec 6 may be used. This strategy may result in the production of monoclonal antibodies with enhanced capacities for recognizing endogenous Siglec 6.

The Siglec 6 protein may be used for generating antibodies. Alternatively, specific regions of the Siglec 6 protein may be selected for generating antibodies. For example, hydrophobicity and hydrophilicity analyses of the Siglec 6 amino acid sequence may be used to identify hydrophilic regions in the Siglec 6 structure. Regions of the Siglec 6 protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Fragments containing these residues are particularly suited in generating specific classes of anti-Siglec 6 antibodies.

Methods for preparing a protein for use as an immunogen and for preparing immunogenic conjugates of a protein with a carrier such as BSA, KLH, or other carrier proteins are well known in the art. In some circumstances, direct conjugation using, for example, carbodiimide reagents may be used; in other instances linking reagents such as those supplied by Pierce Chemical Co., Rockford, Ill., may be effective. Administration of a Siglec 6 immunogen is conducted generally by injection over a suitable time period and with use of a suitable adjuvant, as is generally understood in the art. During the immunization schedule, titers of antibodies can be taken to determine adequacy of antibody formation. The Siglec 6 immunogen may be administered to an animal, preferably a non-human animal, using well-known and routine protocol.

While the polyclonal antisera produced as described above may be satisfactory for some applications, for pharmaceutical compositions, monoclonal antibody preparations are generally preferred. In some cases, immortalized cell lines which secrete a desired monoclonal antibody may be prepared using the standard method of Kohler and Milstein or modifications which effect immortalization of lymphocytes or spleen cells, as is generally known. The immortalized cell lines secreting the desired antibodies are screened by immunoassay in which the antigen is the Siglec 6 protein or Siglec 6 -fragment. When the appropriate immortalized cell culture secreting the desired antibody is identified, the cells can be cultured either in vitro or by production in ascites fluid.

Monoclonal antibodies may also be prepared by the trioma technique, the human B-cell hybridoma technique (Kozbor et al. 1983, Immunology Today, 4:72), and the EBV-hybridoma technique (Cole et al., Monoclonal Antibodies and Cancer Therapy, p77-96, Alan R. Liss, Inc., 1985).

The desired monoclonal antibodies are then recovered from the culture supernatant or from the ascites supernatant. Fragments of the monoclonal antibodies of the invention or the polyclonal antisera (e.g., Fab, F(ab′)₂, Fv fragments, fusion proteins) which contain the immunologically significant portion (i.e., a portion that recognizes and binds Siglec 6) can be used as antagonists, as well as the intact antibodies. Fully human or humanized antibodies directed against Siglec 6 are also useful. As used herein, a humanized Siglec 6 antibody is an immunoglobulin molecule which is capable of binding to Siglec 6 and which comprises a FR region having substantially the amino acid sequence of a human immunoglobulin and a CDR having substantially the amino acid sequence of non-human immunoglobulin or a sequence engineered to bind Siglec 6. Methods for humanizing murine and other non-human antibodies by substituting one or more of the non-human antibody CDRs for corresponding human antibody sequences are well known (Jones et al., 1986, Nature 321: 522-525, Riechmnan et al., 1988, Nature 332: 323-327, Verhoeyen et al., 1988, Science 239: 1534-1536). See also, Carter et al., 1993, Proc. Natl. Acad. Sci. USA 89: 4285), and Sims et al., J. Immunol., 1993, 151: 2296.

Use of immunologically reactive fragments, such as the Fab, Fab′, or F(ab′)₂ fragments is often preferable, especially in a therapeutic context, as these fragments are generally less immunogenic than the whole immunoglobulin. Further, bi-specific antibodies specific for two or more epitopes may be generated using methods generally known in the art. Further, antibody effector functions may be modified so as to enhance the therapeutic effect of Siglec 6 antibodies on cancers. For example, cysteine residues may be engineered into the Fc region, permitting the formation of interchain disulfide bonds and the generation of homodimers which may have enhanced capacities for internalization, ADCC and/or complement-mediated cell killing (Caron et al., 1992, J. Exp. Med. 176: 1191-1195; Shopes, J. Immunol., 1992, 148: 2918-2922). Homodimeric antibodies may also be generated by cross-linking techniques known in the art (e.g., Wolff et al., Cancer Res. 53: 2560-2565). The present invention provides pharmaceutical compositions comprising monoclonal Siglec 6 antibodies, anti-idiotypic monoclonal Siglec 6 antibodies or fragments thereof.

The antibodies or fragments may also be produced, using current technology, by recombinant means. Regions that bind specifically to the desired regions of the Siglec 6 protein can also be produced in the context of chimeric or CDR grafted antibodies of multiple species origin. The invention includes an antibody, e.g., a monoclonal antibody which competitively inhibits the immunospecific binding of any of the monoclonal antibodies of the invention to Siglec 6.

Reactivity of anti-Siglec 6 mAbs against the target antigen may be established by a number of well known means, including Western blot, immunoprecipitation, ELISA, and FACS analyses using, as appropriate, Siglec 6 proteins, peptides, Siglec 6-expressing cells or extracts thereof. Anti-Siglec 6 mAbs may also be characterized in various in vitro assays, including complement-mediated tumor cell lysis, antibody-dependent cell cytotoxicity (ADCC), antibody-dependent macrophage-mediated cytotoxicity (ADMMC), tumor cell proliferation, etc. The antibody or fragment thereof of the invention may also be cytostatic to the cell, to which it binds. It is intended that the term “cytostatic” means that the antibody can inhibit growth of, but not necessarily kill, Siglec 6-positive cells.

The Siglec 6 antibodies may be generated using single lymphocyte antibody methods based on the molecular cloning and expression of immunoglobulin variable region cDNAs generated from single lymphocytes that were selected for the production of specific antibodies such as described by Babcook, J. et al. 1996, Proc. Natl. Acad. Sci. USA 93(15):7843-7848 and in WO 92/02551, which is herein incorporated by reference in its entirety. The Siglec 6 antibodies may also be generated using various phage display methods known in the art and include those disclosed by Brinkman et al. (in J. Immunol. Methods, 1995, 182:41-50), Ames et al. (J. Immunol. Methods, 1995, 184:177-186), Kettleborough et al. (Eur. J. Immunol. 1994, 24:952-958), Persic et al. (Gene, 1997, 187:9-18); Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108, all of which are herein incorporated by reference in their entirety. Techniques for the production of single chain antibodies, such as those described in U.S. Pat. No. 4,946,778, which is herein incorporated by reference in its entirety, can also be adapted to produce single chain antibodies to Siglec 6. Also, trangenic mice, or other organisms, including other mammals may be used to express humanized antibodies.

Multivalent Antibodies

The Siglec 6 antibodies or fragments of the present invention may be monovalent, but preferably they are multivalent. The Siglec 6 antibodies may also be single chain antibodies. A single-chain antibody (scFV) can be engineered as described in, for example, Colcher et al., 1999 Ann. N Y Acad. Sci. 880:263-80; and Reiter, 1996, Clin. Cancer Res. 2:245-52. The single chain antibody can be dimerized or multimerized to generate multivalent antibodies having specificities for different epitopes of the same target Siglec 6 protein. By “multivalent” is meant antibody which is at least divalent, that is, has at least two antigen binding sites.

A simple method to produce multivalent antibodies is to mix the antibodies or fragments in the presence of glutaraldehyde. The initial Schiff base linkages can be stabilized, e.g., by borohydride reduction to secondary amines. A diiosothiocyanate or carbodiimide can be used in place of glutaraldehyde as a non-site-specific linker.

The simplest form of a multivalent, multispecific antibody is a bispecific antibody. Bispecific antibodies may be made by methods known in the art (Milstein et al., 1983, Nature 305:537-539; WO 93/08829; Traunecker et al., 1991, EMBO J., 10:3655-3659, which are herein incorporated by reference in their entireties). Bispecific antibodies can be made by a variety of conventional methods, e.g., disulfide cleavage and reformation of mixtures of whole IgG or, preferably F(ab′)₂ fragments, fusions of more than one hybridoma to form polyomas that produce antibodies having more than one specificity, and by genetic engineering. Bispecific antibodies have been prepared by oxidative cleavage of Fab′ fragments resulting from reductive cleavage of different antibodies. This is advantageously carried out by mixing two different F(ab′)₂ fragments produced by pepsin digestion of two different antibodies, reductive cleavage to form a mixture of Fab′ fragments, followed by oxidative reformation of the disulfide linkages to produce a mixture of F(ab′)₂ fragments including bispecific antibodies containing a Fab′ portion specific to each of the original epitopes. General techniques for the preparation of multivalent antibodies may be found, for example, in Nisonhoff et al., 1961, Arch. Biochem. Biophys. 93: 470; Hammerling et al., 1968, J. Exp. Med. 128: 1461, and U.S. Pat. No. 4,331,647, which are herein incorporated by reference in their entireties.

More selective linkage can be achieved by using a heterobifunctional linker such as maleimide-hydroxysuccinimide ester. Reaction of the ester with an antibody or fragment will derivatize amine groups on the antibody or fragment, and the derivative can then be reacted with, e.g., an antibody Fab fragment having free sulfhydryl groups (or, a larger fragment or intact antibody with sulfhydryl groups appended thereto by, e.g., Traut's Reagent). Such a linker is less likely to crosslink groups in the same antibody and improves the selectivity of the linkage.

It is advantageous to link the antibodies or fragments at sites remote from the antigen binding sites. This can be accomplished by, e.g., linkage to cleaved interchain sulfydryl groups, as noted above. Another method involves reacting an antibody having an oxidized carbohydrate portion with another antibody which has at lease one free amine function. This results in an initial Schiff base (imine) linkage, which is preferably stabilized by reduction to a secondary amine, e.g., by borohydride reduction, to form the final product. Such site-specific linkages are disclosed, for small molecules, in U.S. Pat. No. 4,671,958, and for larger addends in U.S. Pat. No. 4,699,784, both of which are incorporated by reference in their entireties.

The interchain disulfide bridges of the antibody F(ab′)₂ fragment having target specificity are gently reduced with cysteine, taking care to avoid light-heavy chain linkage, to form Fab′-SH fragments. The SH group(s) is(are) activated with an excess of bis-maleimide linker (1,1′-(methylenedi-4,1-phenylene)bis-malemide). A Siglec 6-specific Mab, is converted to Fab′-SH and then reacted with the activated target-specific Fab′-SH fragment to obtain a bispecific antibody.

Alternatively, such bispecific antibodies can be produced by fusing two hybridoma cell lines that produce anti-target Mab and anti-Siglec 6 Mab. Techniques for producing tetradomas are described, for example, by Milstein et al., 1983, Nature 305: 537 and Pohl et al., 1993, Int. J. Cancer 54: 418.

Finally, such bispecific antibodies can be produced by genetic engineering. For example, plasmids containing DNA coding for variable domains of an anti-target Mab can be introduced into hybridomas that secrete Siglec 6 antibodies. The resulting “transfectomas” produce bispecific antibodies that bind target and Siglec 6. Alternatively, chimeric genes can be designed that encode both anti-target and anti-Siglec 6 binding domains. General techniques for producing bispecific antibodies by genetic engineering are described, for example, by Songsivilai et al., 1989, Biochem. Biophys. Res. Commun. 164: 271; Traunecker et al., 1991, EMBO J. 10: 3655; and Weiner et al., 1991, J. Immunol. 147: 4035.

A higher order multivalent, multispecific molecule can be obtained by adding various antibody components to a bispecific antibody, produced as above. For example, a bispecific antibody can be reacted with 2-iminothiolane to introduce one or more sulfhydryl groups for use in coupling the bispecific antibody to a further antibody derivative that binds to the same or a different epitope of the target antigen, using the bis-maleimide activation procedure described above. These techniques for producing multivalent antibodies are well known to those of skill in the art. See, for example, U.S. Pat. No. 4,925,648, and Goldenberg, international publication No. WO 92/19273, which are incorporated by reference in their entireties.

Chimeric Antibodies

The pharmaceutical compositions of the present invention may comprise chimeric Siglec 6 antibodies or fragments thereof. Chimeric antibodies are antibodies encoded by immunoglobulin genes that have been genetically engineered so that the light and heavy chain genes are composed of immunoglobulin gene segments belonging to different species. Chimeric antibodies are likely to be less antigenic.

The antigen combining region (variable region) of a chimeric antibody can be derived from a non-human source (e.g. murine) and the constant region of the chimeric antibody which confers biological effector function to the immunoglobulin can be derived from a human source. The chimeric antibody should have the antigen binding specificity of the non-human antibody molecule and the effector function conferred by the human antibody molecule.

In general, the procedures used to produce chimeric Siglec 6 antibodies or fragments thereof may involve the following steps: a) identifying and cloning the correct gene segment encoding the antigen binding portion of the antibody molecule; this gene segment (known as the VDJ, variable, diversity and joining regions for heavy chains or VJ, variable, joining regions for light chains or simply as the V or variable region) may be in either the cDNA or genomic form; b) cloning the gene segments encoding the constant region or desired part thereof, c) ligating the variable region with the constant region so that the complete chimeric antibody is encoded in a form that can be transcribed and translated; d) ligating this construct into a vector containing a selectable marker and gene control regions such as promoters, enhancers and poly(A) addition signals; e) amplifying this construct in bacteria; f) introducing this DNA into eukaryotic cells (transfection) most often mammalian lymphocytes; g) selecting for cells expressing the selectable marker, h) screening for cells expressing the desired chimeric antibody; and i) testing the antibody for appropriate binding specificity and effector functions.

Antibodies of several distinct antigen binding specificities have been manipulated by these protocols to produce chimeric proteins. Likewise, several different effector functions have been achieved by linking new sequences to those encoding the antigen binding region. Some of these include enzymes, immunoglobulin constant regions from another species and constant regions of another immunoglobulin chain. Additionally, procedures for modifying antibody molecules and for producing chimeric antibody molecules using homologous recombination to target gene modification have been described (Fell et al., 1989, Proc. Natl. Acad. Sci. USA 86:8507-8511).

Human and Humanized Antibodies

The pharmaceutical compositions of the present invention may also comprise human or humanized Siglec 6 antibodies. In some circumstances, humanized antibodies are preferred because methods for immunizing animals yield antibody which is not of human origin and the antibodies could elicit an adverse effect if administered to humans. This is also true if the antibodies are to be administered to any other species which is different from the species of origin of the antibodies. As used herein, “humanization” refers to modifying the species-specific region of the antibody to be homologous to the species to be treated. A humanized antibody is one in which only the antigen-recognized sites, or complementarity-determining hypervariable regions (CDRs) are of non-human origin, whereas all framework regions (FR) of variable domains are products of human genes. These “humanized” antibodies present a less xenografic rejection stimulus when introduced to a human recipient.

The framework region could be from a human immunoglobulin molecule (see U.S. Pat. No. 5,585,089). Antibodies include immunoglobulin molecules and immunologically active portions of immunoglobulin molecules, i.e., molecules that contain an antigen binding site that specifically binds an antigen. The immunoglobulin molecules of the invention can be of any class (e.g. IgG, IgE, IgM, IgD, and IgA) or subclass or immunoglobulin molecule.

Methods for producing fully human monoclonal antibodies, including phage display and transgenic methods, are known and may be used for the generation of human mAbs (Vaughan et al., 1998, Nature Biotechnology 16: 535-539; Sanz et al., 2004, Trends Immunol. 25: 85-91). For example, fully human anti-Siglec 6 monoclonal antibodies may be generated using cloning technologies employing large human Ig gene combinatorial libraries (i.e., phage display)(Griffiths and Hoogenboom, 1993, Building an in vitro immune system: human antibodies from phage display libraries, In: Protein Engineering of Antibody Molecules for Prophylactic and Therapeutic Applications in Man. Clark, M. (Ed.), Nottingham Academic, pp 45-64; Burton and Barbas, Human Antibodies from combinatorial libraries. Id., pp 65-82). Fully human anti-Siglec 6 monoclonal antibodies may also be produced using transgenic mice engineered to contain human immunoglobulin gene loci as described in PCT Patent Application WO98/24893, which is herein incorporated by reference in its entirety (see also, Jakobovits, 1998, Exp. Opin. Invest. Drugs 7: 607-614). This method avoids the in vitro manipulation required with phage display technology and efficiently produces high affinity authentic human antibodies.

Another way to accomplish humanization of a selected mouse monoclonal antibody is the CDR grafting method described by Daugherty, et al., 1991, Nucl. Acids Res., 19:2471-2476. Briefly, the variable region DNA of a selected animal recombinant anti-idiotypic ScFv is sequenced by the method of Clackson, et al., 1991, Nature, 352:624-688. Using this sequence, animal CDRs are distinguished from animal framework regions (FR) based on locations of the CDRs in known sequences of animal variable genes. (Kabat et al., Sequences of Proteins of Immunological Interest, 4th Ed. (U.S. Dept. Health and Human Services, Bethesda, Md., 1987)). Once the animal CDRs and FR are identified, the CDRs are grafted onto human heavy chain variable region framework by the use of synthetic oligonucleotides and polymerase chain reaction (PCR) recombination. Codons for the animal heavy chain CDRs, as well as the available human heavy chain variable region framework, are built in four (each 100 bases long) oligonucleotides. Using PCR, a grafted DNA sequence of 400 bases is formed that encodes for the recombinant animal CDR/human heavy chain FR protection.

Siglec 6 Antibody Fusion Protein and Conjugation with Siglec 6 Antibody

The present invention also provides pharmaceutical composition comprising an effective amount of a Siglec 6 antibody fusion protein to treat or prevent diseases associated with mast cell function which may be triggered by inappropriate cellular responses such as but not limited to inappropriate production of IgE or an imbalance of antibody types or Th2 T cell responses. The Siglec 6 antibody or fragment thereof could be fused via a covalent bond, i.e. peptide bond, at the N-terminus or the C-terminus, to another protein or a portion thereof. The portion thereof could be 10, 20, 30, or 50 amino acids. The Siglec 6 antibody or fragment thereof may be linked to the other protein at the N-terminus of the constant domain of the antibody. The Siglec 6 antibody fusion protein may increase in vivo half-life of the protein it is fused.

The Siglec 6 antibody fusion protein comprising Siglec 6 antibody or portion thereof linked to a second protein or peptide may be prepared by standard chemical or recombinant procedures, in which the antibody or fragment thereof is linked either directly or via a coupling agent to the second protein or peptide either before or after reaction with an appropriate polymer. Particular chemical procedures include, for example, those described in WO 93/62331, WO 92/22583, WO 90/195, and WO 89/1476, all of which are herein incorporated by reference in their entireties. Alternatively, the second protein may be linked using recombinant DNA methods, for example as described in WO 86/01533 and EP 0392745, both of which are herein incorporated by reference in their entireties.

The pharmaceutical composition of the present invention may comprise an effective amount of Siglec 6 antibodies or fragments thereof, specifically the antigen binding region, conjugated to a second protein to treat or prevent diseases associated with mast cell function that may be induced by an aberrant biological process. In such a situation, the Siglec 6 antibody or fragment thereof is joined to at least a functionally active portion of a second moiety having therapeutic activity, such as a cytotoxic agent, a radionuclide, or drug moiety to modify a given biological response.

The second moiety may be a therapeutic agent. The therapeutic agent may be a protein or polypeptide possessing a desired biological activity. Examples of such moieties include but are not limited to a toxin, such as abrin, ricin A, pseudomonas exotoxin, or diphtheria toxin, a protein such as tumor necrosis factor, α-interferon, β-interferon, nerve growth factor, platelet derived growth factor or tissue plasminogen activator, a thrombotic agent or an antiangiogenic agent, e.g. angiostatin or endostatin, or a biological response modifier such as a lymphokine, IL-1, IL-2, IL-6, granulocyte macrophage colony stimulating factor (GM-CSF), granulocyte colony stimulating factor (G-CSF), nerve growth factor, or other growth factor.

Therapeutic agents also include cytotoxins or cytotoxic agents including any agent that is detrimental to (e.g. kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, but are not limited to, antimetabolites, such methotrexate, 6-mercaptopurine, 6-thioguanine, cytarabine, 5-fluorouracil decarbazine; alkylating agents, such as mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin; anthracyclines, such as daunorubicin (daunomycin) and doxorubicin; antibiotics, such as dactinomycin (actinomycin), bleomycin, mithramycin, anthramycin (AMC), calicheamicins, or duocarmycins; and antimitotic agents, such as vincristine and vinblastine. Other cytotoxic agents include modeccin A chain, alpha-sarcin, gelonin, mitogellin, retstrictocin, phenomycin, enomycin, curicin, crotin, calicheamicin, sapaonaria officinalis inhibitor, and maytansinoids.

Other therapeutic moieties may include radionuclides, such as 111In, 90Y, Lu177, Bismuth213, Californium252, Iridium192 and Tungsten188/Rhenium188; or drugs, such as but not limited to, alkylphophocholines, topoisomerase I inhibitors, taxoids and suramin.

Techniques for conjugating or joining therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); and Thorpe et al., Immunol. Rev., 62:119-58 (1982)). The Siglec 6 antibodies or fragments thereof for use in this invention include analogs and derivatives that are modified, for example but without limitation, by covalent attachment of any type of molecule. Preferably, said attachment does not impair immunospecific binding. In one aspect, the Siglec 6 antibody can be conjugated to a second antibody to form an antibody heteroconjugate (see U.S. Pat. No. 4,676,980, which is herein incorporated by reference in its entirety).

Further, the invention provides Siglec 6 antibody or fragment thereof linked to an enzyme. The enzyme may be capable of converting a prodrug into a cytotoxic drug.

Siglec 6 antibody or fragment thereof may be attached to polyethyleneglycol (PEG) moieties. In one embodiment, a modified Fab fragment is PEGylated, i.e. has PEG covalently attached thereto, e.g. according to the method disclosed in EP-A-0948544, which is herein incorporated by reference in its entirety (see “Polyethyleneglycol Chemistry, Biotechnical and Biomedical Applications,” 1992, J. Milton Harris (ed), Plenum Press, New York; “Polyethyleneglycol Chemistry and Biological Applications,” 1997, J. Milton Harris and S. Zalipsky (eds), American Chemical Society, Washington D.C.; and “Bioconjugation protein Coupling Techniques for the Biomedical Sciences,” 1998, M. Aslam and A. Dent, Grove Publishers, New York Chapman, A. 2002, Advanced Drug Delivery Reviews 2002, 54:531-545). In another embodiment, a PEG modified Fab fragment has a maleimide group covalently linked to a single thiol group in a modified hinge region. A lysine residue may be covalently linked to the maleimide group. To each of the amine groups on the lysine residue may be attached a methoxypolyethyleneglycol polymer having a molecular weight of about 20 Kda.

Pharmaceutical Formulations

The present invention provides pharmaceutical compositions comprising a therapeutically effective amount of Siglec 6 antibodies or fragments thereof and a pharmaceutically acceptable carrier, for instance, a carrier suitable for human use. The pharmaceutical compositions are administered at a concentration that is therapeutically effective to treat or prevent diseases or disorders associated with mast cell functions, such as urticaria, atopic dermatitis, or asthma. To accomplish this goal, the compositions may be formulated using a variety of acceptable excipients known in the art. Typically, the compositions are administered by injection, either intravenously or intraperitoneally. Methods to accomplish this administration are known to those of ordinary skill in the art. It may also be possible to obtain compositions which may be topically or orally administered, or which may be capable of transmission across mucous membranes.

Before administration to patients, formulants may be added to the antibodies. A liquid formulation is preferred. For example, these formulants may include oils, polymers, vitamins, carbohydrates, amino acids, salts, buffers, albumin, surfactants, or bulking agents. Preferably carbohydrates include sugar or sugar alcohols such as mono, di, or polysaccharides, or water soluble glucans. The saccharides or glucans can include fructose, dextrose, lactose, glucose, mannose, sorbose, xylose, maltose, sucrose, dextran, pullulan, dextrin, α and β cyclodextrin, soluble starch, hydroxethyl starch and carboxymethylcellulose, or mixtures thereof. Sucrose is most preferred. “Sugar alcohol” is defined as a C₄ to C₈ hydrocarbon having an OH group and includes galactitol, inositol, mannitol, xylitol, sorbitol, glycerol, and arabitol. Mannitol is most preferred. These sugars or sugar alcohols mentioned above may be used individually or in combination. There is no fixed limit to amount used as long as the sugar or sugar alcohol is soluble in the aqueous preparation. Preferably, the sugar or sugar alcohol concentration is between 1.0 w/v % and 7.0 w/v %, more preferable between 2.0 and 6.0 w/v %. More preferably amino acids include levorotary (L) forms of camitine, arginine, and betaine; however, other amino acids may be added. Preferred polymers include polyvinylpyrrolidone (PVP) with an average molecular weight between 2,000 and 3,000, or polyethylene glycol (PEG) with an average molecular weight between 3,000 and 5,000. It is also preferred to use a buffer in the composition to minimize pH changes in the solution before lyophilization or after reconstitution. Most any physiological buffer may be used, but citrate, phosphate, succinate, and glutamate buffers or mixtures thereof are preferred. Most preferred is a citrate buffer. Preferably, the concentration is from 0.01 to 0.3 molar. Surfactants that can be added to the formulation are shown in EP Nos. 270,799 and 268,110, both of which are incorporated by reference in their entireties.

Additionally, antibodies can be chemically modified by covalent conjugation to a polymer to increase its circulating half-life, for example. Preferred polymers, and methods to attach them to peptides, are shown in U.S. Pat. Nos. 4,766,106; 4,179,337; 4,495,285; and 4,609,546 which are all hereby incorporated by reference in their entireties. Preferred polymers are polyoxyethylated polyols and polyethylene glycol (PEG). PEG is soluble in water at room temperature and has the general formula: R(O—CH₂—CH₂)ₙOR where R can be hydrogen, or a protective group such as an alkyl or alkanol group. Preferably, the protective group has between 1 and 8 carbons, more preferably it is methyl. The symbol n is a positive integer, preferably between 1 and 1,000, more preferably between 2 and 500. The PEG has a preferred average molecular weight between 1000 and 40,000, more preferably between 2000 and 20,000, most preferably between 3,000 and 12,000. Preferably, PEG has at least one hydroxy group, more preferably it is a terminal hydroxy group. It is this hydroxy group which is preferably activated to react with a free amino group on the inhibitor. However, it will be understood that the type and amount of the reactive groups may be varied to achieve a covalently conjugated PEG/antibody of the present invention.

Water soluble polyoxyethylated polyols are also useful in the present invention. They include polyoxyethylated sorbitol, polyoxyethylated glucose, polyoxyethylated glycerol (POG), etc. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a preferred molecular weight in the same range as PEG. The structure for POG is shown in Knauf et al., 1988, J. Bio. Chem. 263:15064-15070, and a discussion of POG/IL-2 conjugates is found in U.S. Pat. No. 4,766,106, both of which are hereby incorporated by reference in their entireties.

Another drug delivery system for increasing circulatory half-life is the liposome. Methods of preparing liposome delivery systems are discussed in A. Gabizon et al., Cancer Research, 1982, 42:4734; Cafiso, Biochem. Biophys. Acta., 1981, 649:129; Szoka, Ann. Rev. Biophys. Eng., 1980, 9:467. Other drug delivery systems are known in the art and are described in, e.g., M. J. Poznansky et al., DRUG DELIVERY SYSTEMS (Juliano, ed., Oxford, N.Y. 1980), pp. 253-315; Poznansky, Pharm. Revs. (1984) 36:277.

After the liquid pharmaceutical composition is prepared, it is preferably lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is preferably administered to subjects using those methods that are known to those skilled in the art.

Siglec-6 Antibody Administration

The present invention provides pharmaceutical compositions comprising Siglec 6 antibodies or fragments thereof for treating or preventing diseases associated with mast cell function induced as a response to inappropriate production of IgE, an imbalance of antibody types or Th2 T cell response, or other physiological processes. Such pharmaceutical compositions may be administered orally, parenterally, such as intravascularly (IV), intraarterially (IA), intramuscularly (IM), subcutaneously (SC), intraperitoneally, transdermally, or the like. Administration may in appropriate situations be by transfusion. In some instances, administration may be oral, nasal, rectal, transdermal or aerosol, where the modified polypeptide allows for transfer to the vascular system.

The compositions of this invention may be formulated in a unit dosage form such as a solution, suspension or emulsion, in association with a pharmaceutically acceptable vehicle for administration to a subject. Such vehicles are inherently nontoxic and nontherapeutic. Examples of such vehicles are saline, Ringer's solution, dextrose solution, and Hanks' solution. Nonaqueous vehicles such as fixed oils and ethyl oleate may also be used. A preferred vehicle is 5% dextrose in saline. The vehicle may contain minor amounts of additives such as substances that enhance isotonicity and chemical stability, including buffers and preservatives.

The dosage and mode of administration will depend on the individual. Generally, the compositions are administered so that antibodies are given at a dose between 1 μg/kg and 20 mg/kg, more preferably between 20 μg/kg and 10 mg/kg, most preferably between 1 and 7 mg/kg. Preferably, it is given as a bolus dose, to increase circulating levels by 10-20 fold and for 4-6 hours after the bolus dose. Continuous infusion may also be used after the bolus dose. If so, the antibodies may be infused at a dose between 5 and 20 μg/kg/minute, more preferably between 7 and 15 μg/kg/minute.

Treatment of Diseases

The present invention further provides a method of treating or preventing diseases or disorders associated with aberrant mast cell function comprising administering pharmaceutical compositions comprising Siglec 6 antibodies or fragments thereof to a subject diagnosed with such a disease or disorder. The mast cell function may be triggered by an inappropriate production of IgE or imbalance of antibody types or Th2 T cell responses, or other cellular responses. The subject may be a mammal in need thereof. The mammal may be a human.

Examples of aberrant mast cell functions include degranulation and release of mast cell derived proteins into other tissue, autoantibodies produced against these mast cell specific proteins, and cell apoptosis. These diseases include, but are not limited to allergic diseases, inflammatory disorders, autoimmune disorders, hyperproliferative disorders, hypersensitivity disorders, interstitial cystitis, irritable bowel syndrome, rheumatoid arthritis, asthma (either early onset or chronic), systemic lupus erythematosis (SLS), psoriasis, scleroderma, rhinitis, keratoconjunctivitis, multiple sclerosis, interstitial cystitis, Crohn's disease, fibrosing alveolitis, bolus mastocytosis, urticaria, and chronic liver disease.

In one embodiment of the invention, Siglec 6 antibodies or fragments thereof are used to treat mast cell dependent disease conditions which may also be dependent on IgE, such as asthma, anaphylaxis, and allergic rhinitis (Salvi and Babu, 2000, New Engl. J. Med. 342:1292; Teran, 2000, Immunol. Today 21:235; Marone, 1998, Immunol. Today 19:5; Corrigan, 1999, Clin. Exp. Immunol. 116:1). Siglec 6 antibodies or fragments thereof may also be used to treat other allergic reactions and immunological disorders including late phase allergic reactions, chronic urticaria, and atopic dermatitis by inhibiting chemokine-induced mast cell degranulation and release of histamine.

Asthma is characterized by three features: intermittent and reversible airway obstruction, airway hyperresponsiveness, and airway inflammation (Galli, 1997, J. Exp. Med. 186:343). Asthma involves the following series of events. Inhaled allergens encounter dendritic cells (allergen presenting cells; APCs) that line the airway. The dendritic cells then migrate to lymph nodes, where they present antigen to T cells. Contact of the dendritic cells with the T cells activates the T cells, and once activated, the T cells may produce IL-4 and IL-13 (which act on B cells to promote IgE production) and IL-5 (which recruits eosinophils) (Jaffar, et al. (1999) J. Immunol. 163:6283).

B cells reside in lymph nodes. Two signals are required to provoke B cells to secrete IgE: (1) IL-4 (or IL-13) contact with B cells; and (2) T cell contact with B cells. The occurrence of both of these signals provokes the B cells to produce IgE. The IgE, in turn, circulates in the blood, where it may bind FcεRI of mast cells and basophils, sensitizing the mast cells and basophils, which will then release various inflammatory mediators when they encounter allergen. Mast cells can produce IL-1, IL-2, IL-3, IL-4, IL-5, granulocyte-macrophage stimulating factor, IFN-γ, and TNF-α, histamine, leukotrienes, and reactive oxygen species. Histamine and leukotrienes can provoke smooth muscles to contract, resulting in airway obstruction. IL-5 can recruit eosinophils, and once recruited, the eosinophils may produce “major basic protein,” a protein that can directly damage the airways (Plager, et al., 1999, J. Biol. Chem. 274:14464). The eosinophils also produce leukotrienes, which can provoke the airways to contract.

Environmental allergens initiate the pathway leading to the production of IgE by B cells. These allergens also are used for the cross-linking of IgE/FcεRI complexes residing on the surface of mast cells, where the cross-linking results in mast cell activation. Asthma tends to occur in people who are hypersensitive to specific environmental allergens, such as dust mite allergen, cockroach allergen, pollen, and molds (Barnes, 1999, New Engl. J. Med. 341:2006). In humans, IgE is the main type of immunoglobin (Ig) that mediates airway hypersensitivity (Galli, 1997, J. Exp. Med. 186:343). In fact, there is a strong correlation between serum IgE levels and asthma. IgE is elevated in patients with bronchial asthma and allergic rhinitis (Zuberi, et al., 2000, J. Immunol. 164:2667). Mast cells express receptors (FcεRI) that bind the constant region of IgE antibodies. Injections of recombinant antibodies against IgE have been used to treat asthma. When a mast cell bearing bound IgE molecules encounters an antigen recognized by the bound IgE molecule, the antigen binds, resulting in the mast cell secreting histamine, proteases, prostaglandins, leukotrienes, toxic oxygen, and cytokines. In this situation, the allergen cross-links IgE molecules that are bound to FcεRI, resulting in activation of the mast cell (Kita, et al., 1999, J. Immunol. 162: 6901).

The airways of asthma patients contain accumulations of mast cells, but also of T cells (Th2 type), eosinophils, basophils, and macrophages. Macrophages express FcεRIIB (low affinity IgE receptor), where binding of IgE plus allergen can stimulate the macrophage to release prostaglandins, toxic oxygen, and cytokines (Ten, et al. 1999, J. Immunol. 163:3851).

Clinically, asthma is recognized by airway hyperactivity and reversible airways obstruction. Pathological derangements at the tissue level include constriction of airway smooth muscle, increased vascular permeability resulting in edema of airways, outpouring of mucus from goblet cells and mucus glands, parasympathetic nervous system activation, denudation of airway epithelial lining cells, and influx of inflammatory cells. Underlying these tissue effects are direct effects of potent mediators secreted following physical, inflammatory, or immunological activation and degranulation. The early phase of the asthmatic reaction is mediated by histamine and other mast cell mediators that induce rapid effects on target organs, particularly smooth muscle. The pathophysiologic sequence of asthma may be initiated by mast cell activation in response to allergen binding to IgE. Evidence exists to link exercise-induced asthma and so-called “aspirin-sensitive” asthma to HuMC degranulation.

In another embodiment of the invention, Siglec 6 antibodies and fragments thereof are used to treat inflammatory diseases of the gut, such as inflammatory bowel disease, Crohn's disease (Beutler, 2001, Immunity 15:5; Targan et al., 1997, New Engl. J. Med. 337:1029), colitis (Simpson, et al., 1998, J. Exp. Med. 187:1225), interstitial cystitis, irritable bowel syndrome, and celiac disease. Siglec 6 antibodies and fragments thereof may also be used to treat autoimmune diseases, such as multiple sclerosis, diabetes mellitus, systemic lupus erythematosus (SLE), Sjogren's syndrome, rheumatoid arthritis, scleroderma, polymyositis, autoimmune thyroid disease, autoimmune gastritis and pernicious anemia, and autoimmune hepatitis (Bradley, et al., 1999, J. Immunol. 162:2511; Stott, et al., 1998, J. Clin. Invest. 102:938; Rose and Mackay, 1998, The Autoimmune Diseases, 3. sup. rd ed., Academic Press, San Diego, Calif.).

Moreover, Siglec 6 antibodies or fragments thereof may be used to treat hyperproliferative diseases such as mastocytosis. Mastocytosis may be cutaneous as in solitary mastocytomas or urticaria pigmentosa lesions or it can be systemic where internal organs such as bone marrow, liver and spleen can be affected. In cutaneous mastocytosis, an increased number of mast cells have been seen. Thus, administration of Siglec 6 antibody can potentially inhibit the hyperproliferation of mast cells.

Methods to Identify Agents that Modulate Mast Cell Activation

The present invention further provides a method for identifying agents that modulate mast cell activation or Siglec 6 dependent mast cell function. Agents identified by the disclosed methods are potentially useful for treating diseases and conditions associated with abnormal mast cell function which are induced by inappropriate production of IgE, an imbalance of antibody types or Th2 T cell responses, or other undesirable responses. Such methods or assays may utilize any means of monitoring or detecting the desired activity.

In one embodiment, mast cell activation between a cell population that has been exposed to the agent to be tested is compared to an un-exposed control cell population. Mast cell activation can also be measured between a population that has been exposed to Siglec 6 antibodies and an agent to be tested compared to an un-exposed control cell population. Cell lines or populations are exposed to the agent to be tested under appropriate conditions and time. Mast cells may be exposed to the test agent and Siglec 6 antibodies concurrently or they may be activated prior to incubation with the test agent alone or together with Siglec 6.

Agents that are assayed in the above method can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agents action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant G A. in: Meyers (ed.) Molecular Biology and Biotechnology (New York, VCH Publishers, 1995), pp. 659-664). As used herein, the term “small molecule” refers to compounds having molecular mass of less than 3000 Daltons, preferably less than 2000 or 1500, still more preferably less than 1000, and most preferably less than 600 Daltons. A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Without further description, it is believed that one of ordinary skill in the art can, using the preceding description and the following illustrative examples, make and utilize the claimed invention. The following working examples therefore, specifically point out preferred embodiments of the present invention, and are not to be construed as limiting in any way the remainder of the disclosure. All articles, publications, patents and documents referred to throughout this application are hereby incorporated by reference in their entirety.

### EXAMPLES

### Example 1

### Siglec-6 mRNA Expression in Human Tissues and Inflammatory Cells

This Example provides an analysis of the expression of Siglec 6 expression in various tissues and inflammatory cells.

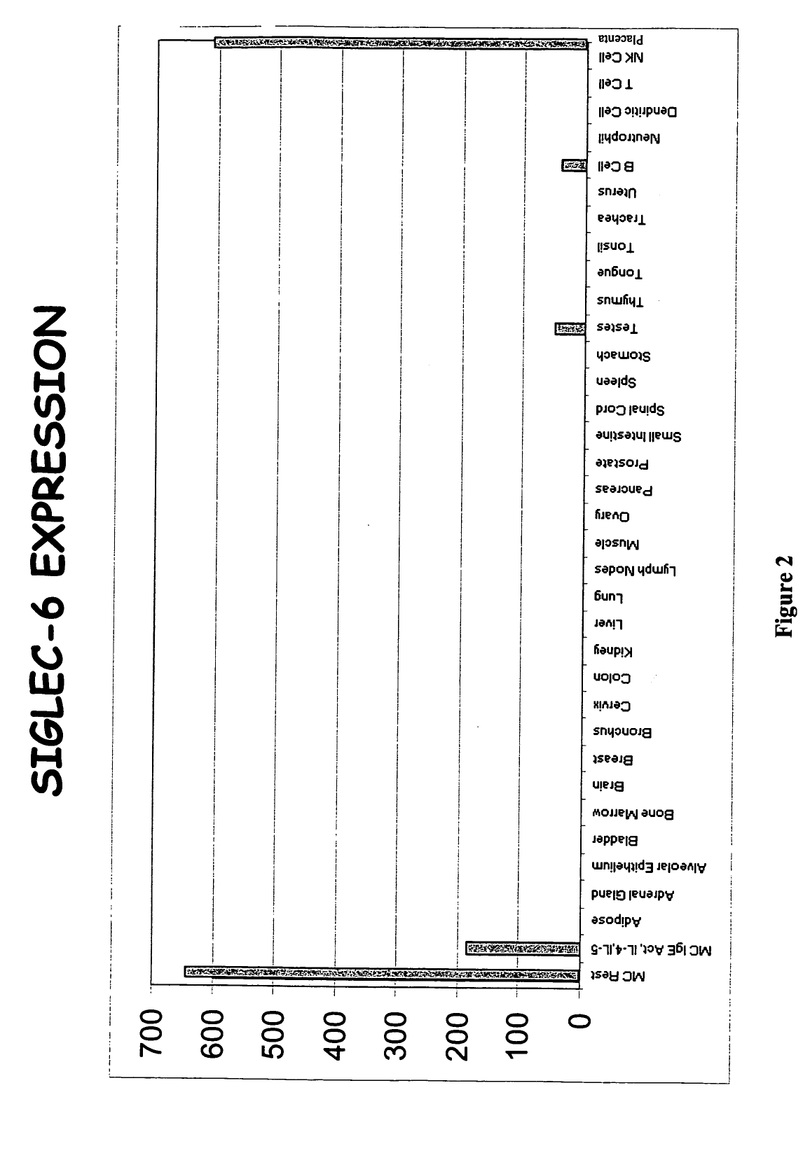

An electronic Northem was performed using an Affymetrix U133 human genome data set to determine Siglec 6 expression in various human tissues and inflammatory cell populations. Electronic northern analysis involved the grouping of RNA sources into organ, tissue, and cell categories. The organ, tissue, and cell categories were adipose, adrenal gland, alveolar epithelium, bladder, bone marrow, brain, breast, bronchus, cervix, colon, kidney, liver, lung, lymph nodes, muscle, ovary, pancreas, prostate, small intestine, spinal cord, spleen, stomach, testes, thymus, tongue, tonsil, trachea, uterus, placenta, B cell, neutrophil, dendritic cell, T cell, and NK cell. The mean expression value of samples in each category are given (FIG. 2)

FIG. 2 shows that Siglec-6 is highly expressed in resting mast cells and modestly expressed in mast cells activated with IgE and antigen in the presence of IL-4 and IL-5. There is also high expression seen in placenta and very low expression in B-cell and testes.

### Example 2

### Inhibition of Mast Cell Activation by IgE and Antigen with Siglec 6 Antibody Crosslinking

This example demonstrates that monoclonal Siglec 6 antibodies inhibit human mast cell activation induced by inflammatory agents.

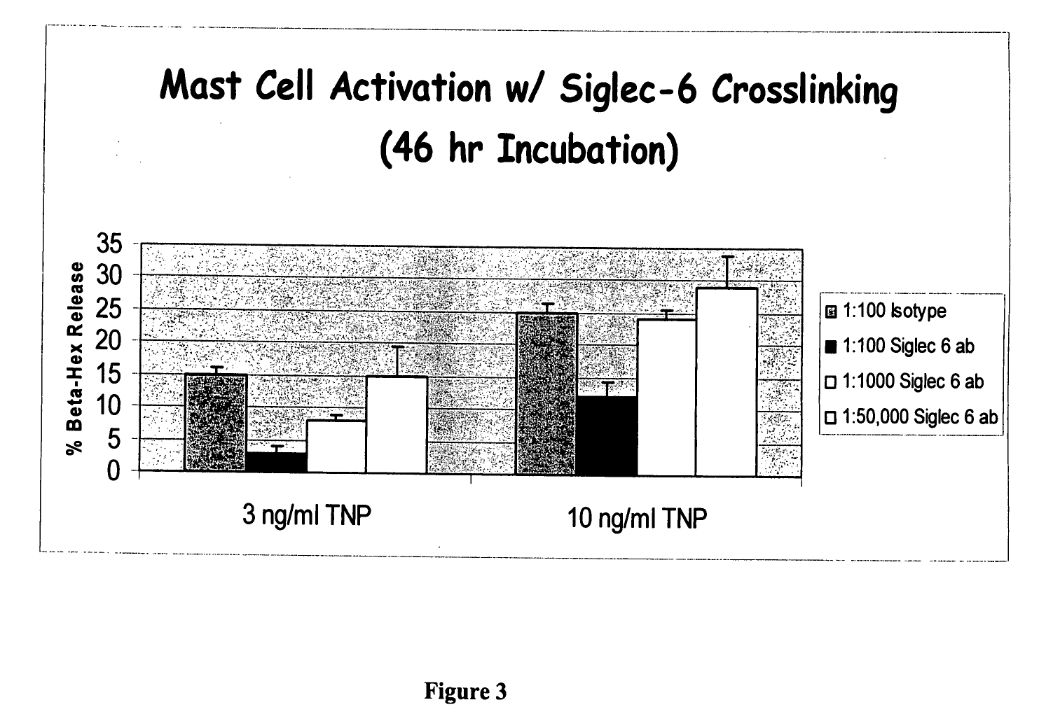

Mast cells were pre-incubated with unconjugated human Siglec 6 monoclonal antibody or an unconjugated isotype matched control antibody for 46 hours. The stock of the antibody was diluted to reach a final concentration of 1:100, 1:1000, and 1:50,000 dilution. After incubation, mast cells were treated for 30 minutes with IgE and antigen, TNP-BSA (2,4,6-Trinitrophenyl hapten conjugated to Bovine Serum Albumin) at concentrations of 3 ng/mL and 10 ng/mL, respectively and the release of β-hexosaminidase from mast cells was measured as an indication of cell activation. As a control for 100% release, Triton X at 1% was used. After incubation, the plate was centrifuged for 5 minutes. 30 μl of supernatant per well was transferred to a 96 well flat bottom plate. After adding 100 μl beta-hexosaminidase substrate solution and incubating 1.5 hours at 37° C., the plate was read at 405 nm. As shown in FIG. 3, about 15% of the total secretory granule enzyme β-hexosaminidase was released from cells incubated with isotype matched antibody and later challenged with 3 ng/ml TNP. When the concentration of TNP was 10 ng/ml, about 25% of β-hexosaminidase was released. Compared to the isotype matched control, release of β-hexosaminidase from mast cells pre-incubated with anti-Siglec 6 antibodies was reduced at both concentrations of TNP. Siglec 6 antibody (1:100 dilution) inhibits the release of β-hexosaminidase by more than 80% when TNP was at a concentration of 3 ng/ml level and more than 55% when the TNP concentration was 10 ng/ml. However, no inhibition was seen when the anti-Siglec 6 antibodies were diluted at a ratio of 1:50,000, suggesting that the inhibition of β-hexosaminidase release was dose-dependent. The inhibitory response was seen after a long period of incubation (46 hour) with the antibody.

### Example 3

### Inhibition of Mast Cell Activation by C3a with Siglec 6 Crosslinking

Human mast cells undergo degranulation in response to the anaphylatoxin, C3a. The above example establishes that anti-Siglec 6 antibody is capable of inhibiting IgE mediated mast cell activation response. This example shows that Siglec 6 crosslinking on human mast cells can also inhibit G protein coupled receptor signaling via C3a.

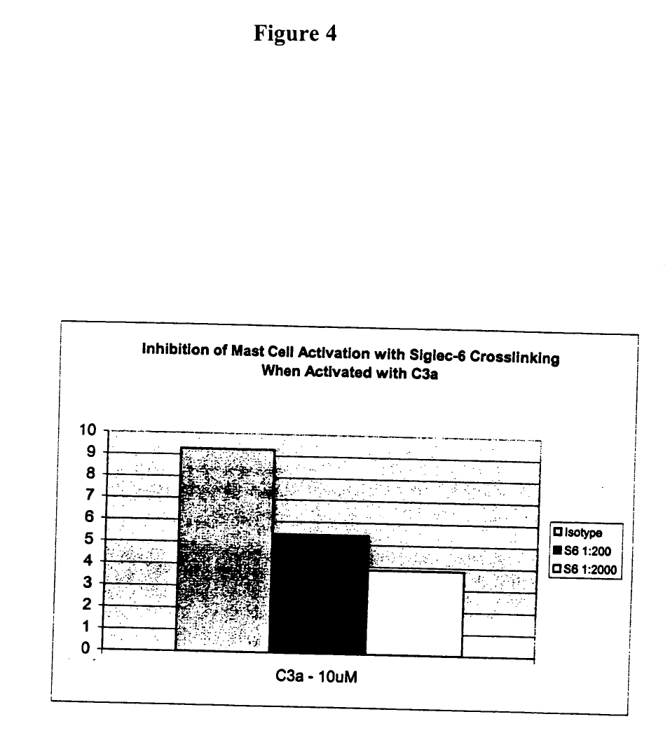

In the example, 6 week old human mast cells were incubated with Siglec 6 unconjugated monoclonal antibodies and unconjugated isotype matched control antibodies for 2 hours at 37° C. and under 5% CO₂. The concentrations of the antibody used were 1:200 dilution for the isotype matched control antibody and 1:200 and 1:2000 dilution for Siglec 6 antibody. After the mast cells were incubated with Siglec 6 antibodies for two hours, cells were washed with IMDM/0.5% BSA+5 ng/mL KL buffer in 37° C. and transferred in aliquot 10,000 cells per well in duplicate to a 96 well plate. After equilibrating the plate for 10 minutes at 37° C., cells were activated with 10 μl of 10 μM C3a for 30 minutes at 37° C. and 5% CO₂. The results presented in FIG. 4 show that while the control antibodies have no effect on the release of β-hexosaminidase, Siglec 6 antibody dose dependently inhibits the release of β-hexosaminidase, suggesting that Siglec 6 crosslinking on human mast cells can inhibit G protein coupled receptor signaling via C3a.

### Example 4

### Inhibition of Mast Cell Activation by Siglec 6 Antibody is Mediated Through Siglec 6

To further confirm that the inhibitory effect of the Siglec 6 antibody was transduced by Siglec 6, interfering RNA (RNAi) was used to knockdown expression of Siglec 6 in human mast cells. It is known that RNAi, usually single oligonucleotides, can be introduced into cells to inhibit gene expression (see U.S. Pat. No. 6,506,559, which is herein incorporated by reference in its entirety). Inhibition of gene expression by RNAi is specific in that a nucleotide sequence from a portion of the target gene is chosen to produce inhibitory RNA.

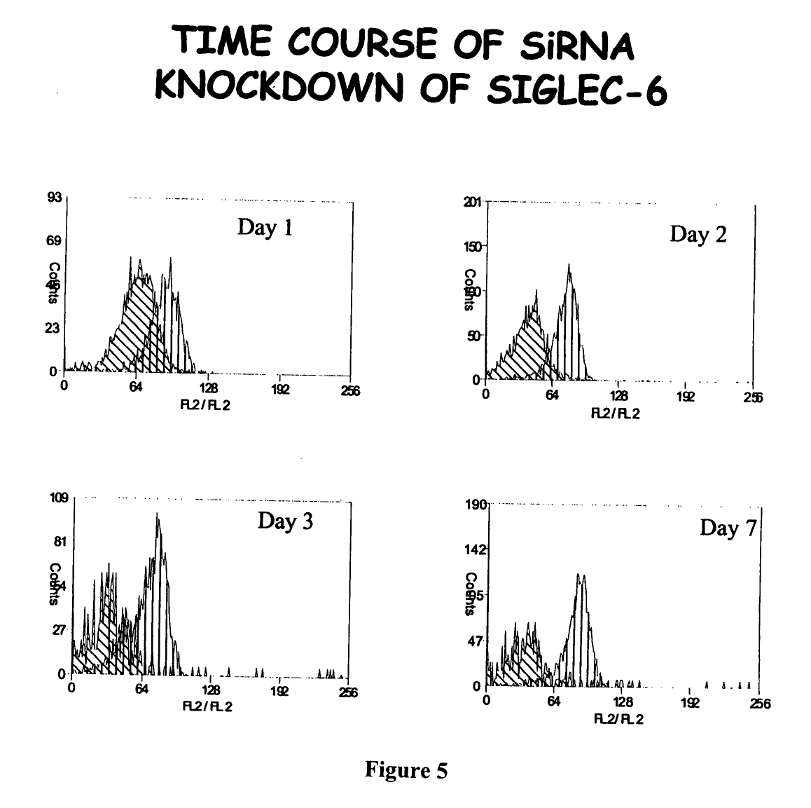

In this example, mast cells were treated for 7 days with single-stranded RNAi oligonucleotides specific for Siglec 6 (cocktail from Dharmacon) or control siRNA. Both cell populations were labeled with a PE-conjugated human monoclonal antibody for siglec-6 and fluorescence was analyzed by FACS for Siglec 6 protein expression on days 1, 2, 3 and 7 after knockdown. FIG. 5 shows the timecourse of siRNA knockdown of siglec-6 expression. Siglec-6 expression on control mast cells is depicted with vertical lines and expression on siRNA treated cells is depicted with diagonal lines. Siglec 6 expression was seen to be lower than control on day 1, decreased to baseline on day 3 and this level of knockdown was sustained out to day 7.

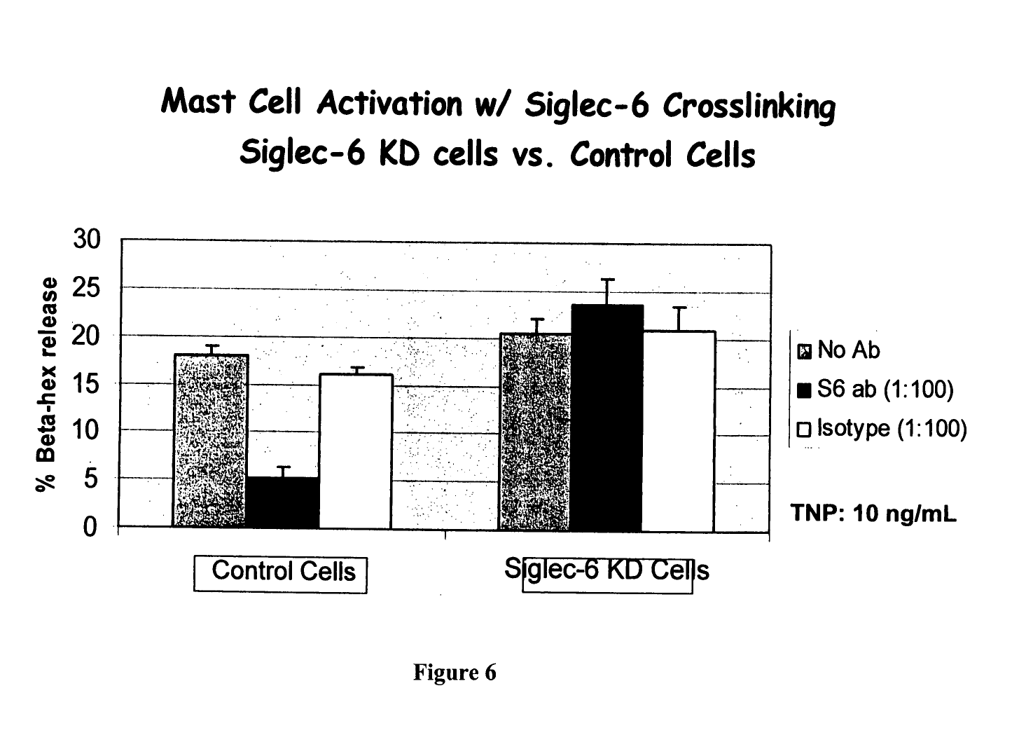

On Day 4, mast cells pretreated with Siglec 6 siRNA and control siRNA were subsequently used for IgE+antigen activation. Mast cells were pre-incubated for 2 hours with either an unconjugated isotype matched control, an unconjugated human siglec-6 monoclonal antibody at a concentration of 1:100 dilution stock, or no antibody treatment. After pre-incubation, cells were activated with IgE+antigen (TNP-BSA, 10 ng/mL) for 30 minutes. β-hexosaminidase release was monitored as compared to unactivated or 100% release (triton-X 100 lysed) controls. Control cells treated with Siglec-6 antibody inhibited beta-hexosaminidase release by about 75% as compared to isotype and no antibody controls. However, no inhibitor of β-hexosaminidase was seen in cells pretreated with Siglec-6 siRNA and later incubated with Siglec-6 antibody and challenged IgE and antigen complex, as shown in FIG. 6. These results demonstrate that the inhibitory effect of Siglec 6 antibody is mediated by protein Siglec 6 as depletion of Siglec 6 expression in human mast cells abolishes the inhibitory activity of the antibody.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are identified in this patent application are incorporated by reference in their entirety.

1. A method of inhibiting mast cell function comprising contacting cells with an effective amount of at least one Siglec 6 antibody or fragment thereof.

2. A method of inhibiting a cellular response associated with mast cell function comprising contacting cells with an effective amount of at least one Siglec 6 antibody or fragment thereof.

3. A method of claim 1, wherein the mast cell function is selected from the group consisting of activation, growth, maturation, proliferation, migration, survival, apoptosis, degranulation, and cytokine release.

4. A method of claim 3, wherein the effective amount of Siglec 6 antibody or fragment thereof inhibits activation of mast cells.

5. A method of claim 4, wherein the effective amount of Siglec 6 antibody or fragment thereof inhibits activation of mast cells by chemotactic factors.

6. A method of claim 5, wherein the chemotactic factor is C3a.

7. A method of claim 3, wherein the effective amount of Siglec 6 antibody or fragment thereof inhibits degranulation of mast cells.

8. A method of claim 7, wherein the degranulation of mast cells is triggered by IgE and antigen.

9. A method of claim 2, wherein the cellular response comprises secretion of mediators from the mast cells.

10. A method of claim 9, wherein the mediators are selected from the group consisting of preformed mediators, newly synthesized mediators, and cytokines.

11. A method of claim 10, wherein the preformed mediators are selected from the group consisting of histamines, proteases, and proteoglycans.

12. A method of claim 10, wherein the newly synthesized mediators are lipid mediators.

13. A method of claim 12, wherein the lipid mediators are selected from the group consisting of leukotriene C4 (LTC4), prostaglandin D2 (PGD2), and platelet activating factor (PAF).

14. A method of claim 10, wherein the cytokines are selected from the group consisting of interleukin-5 (IL-5), interleukin-1β (IL-1β), granulocyte macrophage colony stimulating factor(GMCSF), and tumor necrosis factor-α (TNF-α),

15. A method of treating or preventing a disorder induced by mast cell activation in a subject comprising administering an effective amount of at least one Siglec 6 antibody or fragment thereof to the subject to inhibit the activation of mast cells.

16. A method of claim 15, wherein the disorder is selected from the group consisting of allergic diseases, inflammatory disorders, autoimmune disorders, hyperproliferative disorders, hypersensitivity disorders, interstitial cystitis, and irritable bowl syndrome.

17. A method of claim 16, wherein the allergic disease is urticaria or atopic dermatitis.

18. A method of claim 16, wherein the inflammatory disorder is asthma.

19. A method of claim 16, wherein the hyperproliferative disorder is systemic mastocytosis.

20. A method of targeting a cytotoxic agent to a mast cell comprising contacting the mast cell with a cytotoxic agent comprising a cytotoxin and a Siglec 6 antibody or fragment thereof.

21. A method of depleting mast cells comprising contacting mast cells with a cytotoxic agent comprising a cytotoxin and a Siglec 6 antibody or fragment thereof.

22. A method of identifying an agent that modulates mast cell activation comprising:
a) incubating mast cells expressing Siglec 6 with a test agent;
b) determining whether the test agent modulates at least one Siglec 6 dependent activity, thereby determining whether the test agent modulates mast cell activation.

23. A method of identifying an agent that modulates mast cell activation comprising:
a) incubating mast cells expressing Siglec 6 with a Siglec 6 antibody or fragment thereof, and a test agent;
b) determining whether the test agent modulates Siglec 6 antibody binding, thereby identifying an agent that modulates mast cell activation.

24. A method of claim 22, wherein the mast cells are activated prior to incubation with the test agent.

25. A method of claim 22, wherein the mast cells are activated after incubation with the test agent.

26. A method of claim 1, wherein the Siglec 6 antibody is a bivalent antibody, a trivalent antibody, or a multivalent antibody.

27. A method of claim 1, wherein the antibody is a monoclonal antibody.

28. A method of claim 1, wherein the antibody is a polyclonal antibody.

29. A method of claim 1, wherein the antibody is a chimeric antibody.

30. A method of claim 29, wherein the antibody is a humanized antibody.

31. A method of claim 1, wherein the antibody is a human antibody.

32. A method of claim 1, wherein the antibody further comprises a cytotoxic agent.

33. A pharmaceutical composition comprising a suitable amount of at least one Siglec 6 antibody or fragment thereof and a carrier for administering to humans.
